(19) United States
(12) Patent Application Publication
(10) Pub. No.: US 2012/0309851 A1
(43) Pub. Date: Dec. 6, 2012

(54) Process for Reducing Residual Surface Material from Porous Polymers

(76) Inventors: Nai-Hong Li, Cupertino, CA (US); Yuchiong Hsuanyu, Cupertino, CA (US); James R. Benson, Los Gatos, CA (US); Marc Freed, Aptos, CA (US)

(21) Appl. No.: 13/318,738

(22) PCT Filed: May 4, 2010
(86) PCT No.: PCT/US10/33641

§ 371 Date: Jul. 10, 2012

Related U.S. Application Data
(60) Provisional application No. 61/175,354, filed on May 4, 2009.

(51) Int. Cl. C08F 212/08; C11D 7/26; C08F 220/10; A61K 8/81; C08F 116/06; C11D 7/18; A61K 47/32

(52) U.S. Cl. 514/772.2; 510/244; 514/772.4; 502/159; 521/146; 521/141; 521/149; 514/772.6

(57) ABSTRACT

The present invention relates to methods for removing residual surface material from porous polymerized particle surfaces. The particles thus produced have an increase in surface porosity and uniformity in a variety of applications. Desirably, substantially the entire surface communicates with the interior of the particles. Also provided are the particles produced by such methods, further modifications of such particles, and methods for using the particles in a variety of applications. All described methods, compositions, and articles of manufacture are within the scope of the invention.

### FIELD OF THE INVENTION

The present invention relates to methods for reducing residual surface material from porous cross-linked polymeric material, particles and polymers produced by such techniques, methods for their use, and articles and apparatuses comprising them.

### BACKGROUND OF THE INVENTION

Cross-linked, homogeneous, porous block polymeric materials are disclosed in U.S. Pat. No. 4,522,953 (Barby et al., issued Jun. 11, 1985). The described polymeric materials produced by polymerization of water-in-oil emulsions having a relatively high ratio of water to oil. These emulsions are termed “high internal phase emulsions” and are known in the art as “HIPE” or “HIPEs”, and the resulting polymeric material is referred to as “HIPE polymers”. HIPE polymers as described in Barby comprise an oil continuous phase including a monomer and a cross-linking agent and an aqueous discontinuous phase. Such emulsions are prepared by subjecting the combined oil and water phases to agitation in the presence of an emulsifier, and then initiating polymerization. The polymers are then washed to remove undesired components. The disclosed porous polymers have rigid structures containing cavities interconnected by pores in the cavity walls.

Processes for large-scale production of HIPE polymers are known. For instance, U.S. Pat. No. 5,149,720 (DesMarais et al., issued Sep. 22, 1992) discloses a continuous process for preparing high internal phase emulsions that are suitable for polymerization into absorbent polymers. In addition, a method that facilitates such continuous processes by reducing the curing time of monomers in a HIPE is set forth in U.S. Pat. No. 5,252,619 (Brownscombe et al., issued Oct. 12, 1993).

One problem with many methods of forming HIPE polymeric blocks is that a coating or skin that forms at the interface between the HIPE and the container used for polymerization. (see U.S. Pat. No. 4,522,953, Barby et al., issued Jun. 11, 1985, at column 4, lines 1-6). To produce a permeable block, and hence, to produce a useful product, the coating or skin must be removed. Typically extensive manual grinding methods are used. This results in particle irregularity, along with waste and inconsistency in the resulting material. Additionally, grinding processes waste substantial amounts of polymer.

In U.S. Pat. Nos. by Li et al. (5,583,162; 5,653,922; 5,760,097; 5,863,957; 6,100,306) incorporated herein by reference, HIPE microbeads are described that avoid many of the problems associated with prior art HIPE materials. In particular, these microbeads have a porous, cross-linked, polymeric structure, characterized by cavities joined by interconnecting pores. At least some of the cavities at the interior of each microbead described in these patents communicate with the surface of the particle. However, in some instances such particles can retain some residual surface material after polymerization which can affect their surface porosity and flow characteristics and result in variability between product batches. See FIG. 1.

There is a need in the art for improved methods of removing residual surface material from polymeric materials, and for compositions, articles and devices incorporating such products.

### SUMMARY OF THE INVENTION

The present invention comprises a process for reducing residual surface material on highly porous, cross-linked polymeric particles characterized by cavities joined by interconnecting pores. Desirably, the resulting particles are free from residual surface material on substantially the entire particle surface, and substantially the entire surface communicates with the interior of the particles. See FIG. 2. The particles produced by these methods have an increase in surface porosity and uniformity in a variety of applications. Also provided are the particles produced by this process, methods for their use, and articles and apparatuses comprising them.

More uniform polymeric particles have more desirable properties in a variety of applications, for example permit higher resolution separations as compared to nonuniform particles, and can require less chromatographic packing material for a given separation, thereby permitting more efficient use of such material, as well as more rapid separations. With improved surface porosity, the flow rate through such material is improved, and results in more uniform particles. Furthermore, by providing processes which increase the uniformity of particles, batch to batch variations in different production lots of polymeric materials can be reduced. This provides additional efficiencies in decreasing the amount of experimentation needed to adapt use protocols for different batches of particles.

In some embodiments, methods for improving the surface porosity of a porous polymeric material are provided comprising contacting a polymerized porous material having residual surface material of reduced porosity with a surface material disrupting agent under conditions that permit disruption of the material to occur. The treating material is then recovered, and can be washed. The methods are useful where the residual surface material comprises an erodible component susceptible to disruption by a suitable disrupting agent. In some embodiments, the surface material may comprise amide linkages, and may comprise a protein component, or another biopolymer.

In some embodiments, the surface material disupting agent can take the form of a small molecule. In some embodiments, the surface material disupting agent is selected from a peroxide, an anhydride, or a combination thereof. In some embodiments, the material is treated at an elevated temperature and pH, and may be treated 24 hours or less.

In some embodiments, the polymeric material can be prepared by suspension polymerization, which may be done using an erodible stabilizing agent in the suspension medium. In some embodiments, the polymerized porous material is prepared using an optionally derivatized alkenyl or alkynyl monomer, or a mixture thereof, which may be an optionally derivatized vinyl monomer. In some embodiments, the resulting particle has a void volume of at least 75%, and may be at least 80%, at least 85%, at least 90%, at least 95%, or at least 97%.

The present invention also encompasses modifications of the particles thus produced for use in particular applications. In particular, the present invention includes particles functionalized for absorption of liquids, carbonized particles optionally have a metal deposited within the particle, particles having a gel or pre-gel within the particle cavities and particles having other ingredients or formulations within the particle cavities, as well as processes for producing such particles.

In addition, the present invention includes the use of particles thus produced in a variety of applications that benefit from particles having improved surface porosity, including: the use of particles as a substrate in separation technologies; the use of particles in various solid phase synthesis applications; the use of particles as a substrate for immobilizing a molecule such as a polypeptide, an enzyme, an oligonucleotide or other macromolecule; the use of particles in cell culture methods; the use of particles to contain whole viruses, the use of particles in gene therapy applications; the use of particles as carriers of active ingredients such as pharmaceutical agents; the use of particles as carriers for various cosmetic formulations and skin care applications; the use of particles as a scaffolding for tissue culture applications; the use of particles as a scaffolding for synthetic cartilage; the use of particles as a scaffolding for artificial organs, e.g. the liver; the use of particles to contain various catalysts; the use of particles for fuel cell applications and as conductive materials in a variety of electrochemical conversion processes; the use of particles as carriers for various adhesives; the use of particles as a low-density filler; and the use of particles in conjunction with conductive polymer applications.

### BRIEF DESCRIPTION OF THE DRAWINGS

FIG. 1 is a scanning electron micrograph of a porous polymeric microbead comprising residual surface material occluding a significant percentage of its available surface.

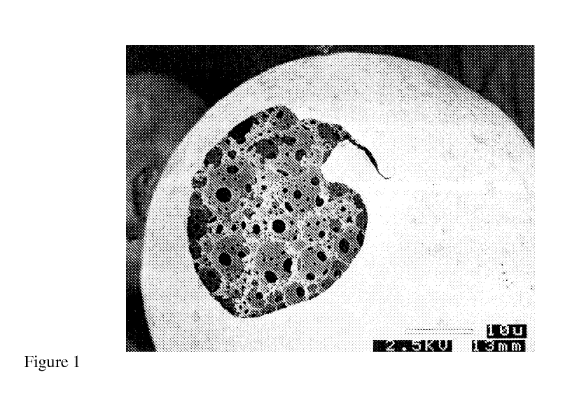

FIG. 2 depicts a HIPE-derived particle that has been treated by a method of the invention to remove residual surface material remaining after polymerization. No remaining residual surface material occluding the surface can be seen. The second step of the process is to add the emulsion to an aqueous suspension medium to form an oil-in-water suspension of dispersed emulsion droplets.

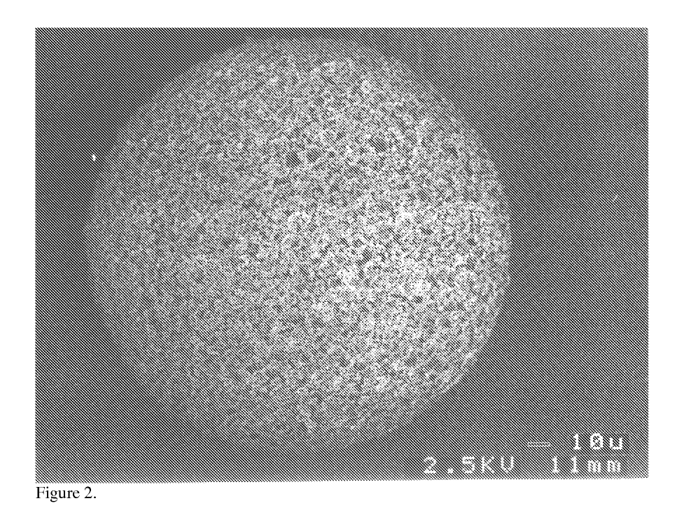

### DETAILED DESCRIPTION OF THE INVENTION

Porous materials can be deleteriously affected by residual surface material when used in a variety of applications. Removing residual surface material can improve particle properties that rely on porosity, including absorption characteristics, flow characteristics, and batch to batch uniformity. Known processes of removing surface polymer from porous materials are tedious and costly. Typically extensive manual grinding methods may be required. This results in particle irregularity, along with waste and inconsistency in the resulting selected material. This limits the efficiency and resolution when used in chromatographic applications. Additionally, grinding and sieving processes waste substantial amounts of polymer.

The present invention provides cross-linked porous polymeric materials with reduced residual surface material, referred to as a “particle” or “particles,” and methods for making them. A particle is produced, for example, by suspension polymerization or by filling a mold form having a predetermined shape with a high internal phase emulsion, termed a “HIPE”. The particle thus has many of the desirable physical characteristics of prior art HIPE polymers (such as those disclosed in U.S. Pat. No. 4,522,953, Barby et al., issued Jun. 11, 1985, which is incorporated by reference herein in its entirety) and the patents of Li et al. as described above and incorporated by reference herein in their entirety. Specifically, the particle has a very low density due to the presence of numerous spherical cavities joined by smaller-diameter interconnecting pores. The void volume of the particle is at least about 70% and, in a preferred embodiment, is at least about 90%. The measured dry density, determined from the weight of a known volume of settled particles, is less than about 0.20 gm/cm³, and in some embodiments less than about 0.10 gm/cm³. This high porosity and low density gives the particle exceptional absorbency. Furthermore, because the interconnectedness of the cavities in the particle allows liquids to flow through the particle, the particle provides an excellent substrate for use in biotechnology and biomedical applications such as, for example, chromatographic separation of biomolecules, and in biomolecule synthesis, in gene therapy applications and as scaffolding for tissue engineering applications.

Where the particles are microbeads formed via suspension polymerization, their average diameter typically ranges from about 10 μm up to about 5 mm. The preferred average diameters range from about 50 μm to about 500 μm. This small size facilitates efficient washing, and produces particles of a substantially uniform size and shape. This allows the wash conditions to be optimized to ensure that each particle in a batch has been thoroughly washed, and allows for consistency between batches.

An important feature is that the particle thus produced is substantially free of residual surface material such that nearly all interior cavities and pores communicate with the surface of the particle. The resulting structures have a series of successive spherical cavities linked by smaller-diameter interconnecting pores extending across the interior of the particles. This feature contributes to improvements in washing of the particles, such that washing solvents can easily flow through the entire volume of the particles. In some embodiments, at least 50%, at least 60%, at least 70%, at least 80%, at least 85%, at least 90%, or at least 95% of the cavities at the interior of the particle communicate with the surface of the particle. This feature of the present invention facilitates cost-efficient scale-up of HIPE polymer production.

Any suitable polymer precursor that can form the particles of interest can be used. For example, the continuous phase may include monomers and cross-linkers as disclosed by Li et al. (above). Of particular interest are derivatized vinyl monomers, e.g. styrene. In some embodiments, divinylbenzene is used as the cross-linking agent, and sorbitan monooleate as the emulsifier. In addition, the continuous phase contains an oil-soluble polymerization initiator such as azoisobisbutyronitrile as well as a material such as dodecane, to promote the formation of interconnecting pores. The aqueous discontinuous phase of at least 70% may include a water-soluble polymerization initiator, e.g. potassium persulfate.

The particles and compositions of this invention offer advantages in applications that benefit from utilizing particles that have substantially porous surfaces. This feature provides improved particles useful as an absorbent material and also as a solid support in a variety of chemical, biotechnology, biomedical and related applications, including chromatographic separations, solid phase synthesis, immobilization of antibodies or enzymes, cell culture and tissue engineering. These particles are also useful in consumer applications such as cosmetics, feminine care, oral care and wound treatment. Moreover, many of the physical characteristics of the particle, such as void volume and cavity size, are controllable. Therefore, different types of particles, specialized for different uses, can be produced.

### DEFINITIONS

Before the present invention is further described, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a monomer” includes a plurality of monomers, reference to “a particle” includes a plurality of such particles, reference to “a cosmetic” includes a plurality of cosmetics, and the like.

Terms such as “connected,” “attached,” “linked,” and the like are used interchangeably herein and encompass direct as well as indirect connection, attachment, or linkage unless the context clearly dictates otherwise, and includes chemical couplings as well as nonchemical binding or other association. Thus, these terms intend that the particles, chemicals, labels, etc., which are “linked” may be physically linked by, for example, covalent chemical bonds, physical forces such van der Waals or hydrophobic interactions, encapsulation, embedding, or the like.

Where a range of values is recited, it is to be understood that each intervening integer value, and each fraction thereof, between the recited upper and lower limits of that range is also specifically disclosed. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. For any element of an invention for which a plurality of options are disclosed, examples of that invention in which each of those options is individually excluded along with all possible combinations of excluded options are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The term “microbeads” refers to a cross-linked porous polymeric material wherein at least about 10% of the particles are substantially spherical and/or substantially ellipsoidal beads when examined via scanning electron microscopy. Preferably at least about 20% and more preferably at least about 50% of this material consists of substantially spherical and/or substantially ellipsoidal beads. Such particles can be conveniently produced via suspension polymerization.

The term “particle” refers to a cross-linked porous polymeric material produced by polymerizing a stabilized high internal phase emulsion, for example in a mold or via suspension polymerization. Where a mold is used, the resulting particle has a predetermined shape reflecting the shape of the mold (e.g., spheroid, ellipsoid, cylindrical, geometric prism, etc.).

As applied to the components of a HIPE, the phrase “substantially oil-soluble” indicates that the indicated component is present in the oil phase at least 95% by weight.

The term “density” or “dry density” refers to the weight per volume of dry, settled, nonswollen porous polymeric particles. For the particles prepared as described herein, the density is less than about 0.20 gm/cm³, and in some embodiments less than about 0.10 gm/cm³. The density of the polymeric particles is determined as follows. An amount of dry, nonswollen polymeric particles is placed in a vessel having a known volume, for example a 10 ml graduated cylinder, and settled by hand tapping, with additional particles added and settled until the particles reach the known volume in the vessel. The weight of the known volume of settled particles is then measured. The resulting measured weight per known volume provides the density of the particles.

The term “void volume” refers to the volume of a porous polymeric particle that does not comprise polymeric material. In other words, the void volume of a particle comprises the total volume of the cavities. Void volume is expressed as a percentage of the total particle volume. The void volume can be measured as follows. Dry, nonswollen porous polymeric particles are placed in a vessel of known volume, for example a 10 ml graduated cylinder, and settled by hand tapping as described above. A measurable amount of nonswelling, nonsolvent oil is added to the vessel, for example from a burette. Because of the strong capillary forces provided by the highly porous particles, the oil is immediately absorbed by the particles. The volume of such oil added to the particles until visible solvent is present in the vessel provides a measurement of the volume of the voids within the particles. For styrene-derived particles, methanol is a suitable nonswelling, nonsolvent oil for measuring the void volume. Another exemplary oil of use is toluene. Suitable nonswelling, nonsolvent oils are known for other polymers and can be determined empirically. Particles prepared as described herein have a void volume of over 70%, and desirably have a void volume of at least 75%, at least 80%, at least 85%, at least 90%, at least 95%, or at least 97%.

The term “cavity size” refers to the average diameter of the cavities present in a particle, as determined by scanning electron microscopy.

The term “porogen” refers to an organic compound that, when included in the continuous phase of a HIPE, promotes the formation of pores connecting the cavities in the polymer formed by the presence of the included discontinuous phase during polymerization. Exemplary porogens include dodecane, toluene, cyclohexanol, n-heptane, isooctane, and petroleum ether. The porogen is typically present in the continuous phase at a concentration in the range of about 10 to about 60 weight percent.

The abbreviation “DVB” refers to “divinylbenzene”; the abbreviation “AIBN” refers to “azoisobisbutyronitrile”; and the abbreviation “PVA” refers to “poly(vinylalcohol)”, which is typically produced by hydrolysis of a polyvinyl ester, e.g. poly(vinylacetate).

### Formation of a High Internal Phase Emulsion (HIPE)

The polymers of the present invention are conveniently produced from a HIPE, which comprises an emulsion of an aqueous discontinuous phase in an oil continuous phase.

The relative amounts of the two HIPE phases are, among other parameters, important determinants of the physical properties of the resulting polymers. In particular, the percentage of the aqueous discontinuous phase affects void volume, density, and cavity size. For the emulsions used to produce preferred particles, the percentage of aqueous discontinuous phase is generally in the range of about 70% to about 98%, more preferably at least 75%, and most preferably at least 80%.

The continuous phase of the emulsion comprises a monomer, a cross-linking agent, and an emulsifier that is suitable for forming a stable emulsion. Any suitable monomer component(s) can be used; for example, those used in known HIPE polymers, and can be a substantially oil-soluble, monofunctional (having a single polymerizable functionality) monomer. Of particular interest are vinyl or derivatized vinyl, derivatized for example with functional groups such as alkyl, aryl, acids, bases, esters, halogens, ethers, alcohols, and combinations of functional groups; suitable monomers are commercially available. In some embodiments, the monomer type is a styrene-based monomer, such as styrene, 4-methylstyrene, 4-ethylstyrene, chloromethyl styrene, 4-t-BOC-hydroxystyrene. The monomer component can be a single monomer type or a mixture of types. The monomer component is typically present in a concentration of about 5% to about 90% by weight of the continuous phase. The concentration of the monomer component is preferably about 15% to about 50% of the continuous phase, more preferably, about 16% to about 38%.

Exemplary monomer reactants used to form the polymer can include vinyl chloride, vinyl acetate, vinyl alcohol, tert-Butyl cinnamate, 1,1-Dichloroethylene, cis-1,3-Dichloropropene, Diethyl trans-cinnamylphosphonate, Divinyl sulfone, N-Ethyl-2-vinylcarbazole, Ethyl vinyl sulfide, Isoamyl cinnamate, Isobutyl cinnamate, 2-Isopropenyl-2-oxazoline, Isopropyl cinnamate, N-Methyl-N-vinylacetamide, 1-(3-Sulfopropyl)-2-vinylpyridinium hydroxide inner salt, Trichlorovinylsilane, (3,5,5-Trimethylcyclohex-2-enylidene)malononitrile, 9-Vinylanthracene, Vinyl bromide, 9-Vinylcarbazole, Vinylcyclohexane, 4-Vinyl-1-cyclohexene, 4-Vinyl-1-cyclohexene 1,2-epoxide, Vinylcyclopentane, 2-Vinyl-1,3-dioxolane, N-Vinylformamide, 1-Vinylnaphthalene, 2-Vinylnaphthalene, Vinylphosphonic acid, N-Vinylphthalimide, 2-Vinylpyridine, 4-Vinylpyridine, 1-Vinyl-2-pyrrolidinone, Vinylsulfonic acid, Vinyltrimethylsilane, 4-Acetoxystyrene, 4-Benzyloxy-3-methoxystyrene, 2-Bromostyrene, 3-Bromostyrene, 4-Bromostyrene, α-Bromostyrene, 4-tert-Butoxystyrene, 4-tert-Butylstyrene, 4-Chloro-α-methylstyrene, 2-Chlorostyrene, 3-Chlorostyrene, 4-Chlorostyrene, 2,6-Dichlorostyrene, 2,6-Difluorostyrene, 1,3-Diisopropenylbenzene, 3,4-Dimethoxystyrene, α,2-Dimethylstyrene, 2,4-Dimethylstyrene, 2,5-Dimethylstyrene, N,N-Dimethylvinylbenzylamine, 2,4-Diphenyl-4-methyl-1-pentene, 4-Ethoxystyrene, 2-Fluorostyrene, 3-Fluorostyrene, 4-Fluorostyrene, 2-Isopropenylaniline, 3-Isopropenyl-α,α-dimethylbenzyl isocyanate, α-Methylstyrene, 3-Methylstyrene, 4-Methylstyrene, 3-Nitrostyrene, 2,3,4,5,6-Pentafluorostyrene, 2-(Trifluoromethyl)styrene, 3-(Trifluoromethyl)styrene, 4-(Trifluoromethyl)styrene, 2,4,6-Trimethylstyrene, 3-Vinylaniline, 4-Vinylaniline, 4-Vinylanisole, 9-Vinylanthracene, 3-Vinylbenzoic acid, 4-Vinylbenzoic acid, 4-Vinylbenzyl chloride, (Vinylbenzyl)trimethylammonium chloride, 4-Vinylbiphenyl, and 2-Vinylnaphthalene.

The cross-linking agent can be selected from a wide variety of substantially oil-soluble, polyfunctional (having more than one polymerization functionality) crosslinkers. Suitable cross-linking agents are known in the art, for example divinyl aromatic compounds, such as divinylbenzene (DVB). Other types of cross-linking agents, such as di- or triacrylic compounds and triallyl isocyanurate, can also be employed. The cross-linking agent can comprise a single type of cross-linker or can be a mixture of different cross-linkers. The cross-linking agent is generally present in a concentration of about 1% to about 90% by weight of the continuous phase. Preferably, the concentration of the cross-linking agent is less than about 35%, and more preferably is less than about 30%. In some embodiments, the cross-linking agent is in the range of about 15% to about 50% of the continuous phase, more preferably, about 16% to about 38%. In some embodiments, the cross-linking agent is present at a concentration of about 16 to about 25%, and may be about 20%, or in the range of about 1 to about 20%.

In addition to a monomer and a cross-linking agent, the continuous phase comprises an oil-soluble emulsifier that promotes the formation of a stable emulsion. The emulsifier can be any nonionic, cationic, anionic, or amphoteric emulsifier or combination of emulsifiers that promotes the formation of a stable emulsion. Suitable emulsifiers are known in the art and include sorbitan fatty acid esters, polyglycerol fatty acid esters, and polyoxyethylene fatty acids and esters. In some embodiments, the emulsifier is sorbitan monooleate (sold as SPAN 80). The emulsifier is generally present at a concentration of about 3% to about 50% by weight of the continuous phase. Preferably, the concentration of the emulsifier is about 10% to about 25% of the continuous phase. More preferably, the concentration is about 15% to about 20%.

In some embodiments, the continuous phase also contains an oil-soluble polymerization initiator and a porogen. The initiator can be any oil-soluble initiator that permits the formation of a stable emulsion, such as an azo initiator or a peroxide initiator. A preferred initiator is azoisobisbutyronitrile (AIBN). In some embodiments, the initiator is selected from the group consisting of AIBN, benzoyl peroxide, lauroyl peroxide, and a VAZO initiator. The initiator can be present in a concentration of up to about 5 weight percent of total polymerizable monomer (monomer component plus cross-linking agent) in the continuous phase. The concentration of the initiator is preferably about 0.5 to about 1.5 weight percent of total polymerizable monomer, more preferably, about 1.2 weight percent.

The porogen can be any organic compound or combination of compounds that permits the formation of a stable emulsion while promoting pore formation without becoming incorporated into the polymer, provided that the compound is a good solvent for the monomers employed. Preferably, the porogen is a poor solvent for the polymer produced. Suitable porogens include dodecane, toluene, cyclohexanol, n-heptane, isooctane, and petroleum ether. A preferred porogen is dodecane. The porogen is generally present in a concentration of about 10 to about 60 weight percent of the continuous phase. The porogen concentration affects the size and number of pores connecting the cavities in the particle. Specifically, increasing the porogen concentration increases the size and number of interconnecting pores; while decreasing the porogen concentration decreases the size and number of pores. Preferably, the porogen concentration is about 25 to about 40 weight percent of the continuous phase. More preferably, the concentration is about 30 to about 35 weight percent.

In some embodiments, the aqueous discontinuous phase of a HIPE comprises a water-soluble polymerization initiator. In these cases, the initiator can be any suitable water-soluble initiator. Such initiators are known and include peroxide compounds such as sodium, potassium, and ammonium persulfates; sodium peracetate; sodium percarbonate and the like. A preferred initiator is potassium persulfate. The initiator is typically present in a concentration of up to about 5 weight percent of the aqueous discontinuous phase. Preferably, the concentration of the initiator is about 0.5 to about 2 weight percent of the aqueous discontinuous phase.

Where the polymers are to be formed into microbeads, the HIPE may be conveniently added to an aqueous suspension medium to form a suspension of HIPE microdroplets, as is known in the art. Polymerization then converts the liquid HIPE microdroplets to solid porous microbeads. Thus, after formation of a HIPE, the HIPE can be added to an aqueous suspension medium to form an oil-in-water suspension. The aqueous suspension medium comprises a suspending agent and a water-soluble polymerization initiator. The suspending agent can be any agent or combination of agents that promotes the formation of a stable suspension of HIPE microdroplets. Typical droplet stabilizers for oil-in-water suspensions include water-soluble polymers such as gelatin, natural gums, cellulose, polyvinylpyrrolidone and poly(vinyl alcohol) (PVA). The latter can be produced by partial (85-92%) hydrolysis of polyvinyl acetate. Also useful are finely-divided, water-insoluble inorganic solids, such as clay, silica, alumina, and zirconia. Two or more different suspending agents can be combined. In some embodiments, a combination of gelatin or PVA (88% hydrolysis) and modified clay or silica particles can be used as suspending agent.

The suspending agent can be present in the aqueous suspension medium in any concentration that promotes the formation of a stable suspension, typically about 0.1 to about 10 weight percent of the aqueous suspension medium. For a preferred combination of suspending agents, a stable suspension is obtained with a PVA concentration of about 0.5% to about 5% and a inorganic solid concentration of about 0.05 to about 0.3% by weight of the aqueous suspension medium.

In addition to a suspending agent, the aqueous suspension medium can contain a water-soluble polymerization initiator. The presence of an initiator in the suspension medium, as well as in the HIPE microdroplets, accelerates the polymerization reaction. Generally, rapid polymerization is desirable. The initiator can be any suitable water-soluble initiator such as those described above for the aqueous discontinuous phase of the HIPE. In a preferred embodiment, the initiator is potassium persulfate, present in the suspension medium at a concentration of up to about 5 weight percent. More preferably, the concentration of the initiator is about 0.5% to about 2% by weight of the aqueous suspension medium.

The first step in the production of a HIPE-based particle is the formation of a high internal phase emulsion. A HIPE can be prepared by any available method, for example as disclosed in U.S. Pat. No. 4,522,953 (Barby et al., issued Jun. 11, 1985). Briefly, a HIPE is formed by combining the continuous and aqueous discontinuous phases while subjecting the combination to shear agitation. Generally, a mixing or agitation device such as a pin impeller is used.

The extent and duration of shear agitation must be sufficient to form a stable emulsion. As shear agitation is inversely related to cavity size, the agitation can be increased or decreased to obtain a particle with smaller or larger cavities, respectively. By selecting the appropriate stirrer speed and resulting viscosity of the emulsion, n the size of the cavities in the cross-linked polymer can be closely controlled. In some embodiments, a HIPE is prepared using a Gifford-Wood Homogenizer-Mixer (Model 1-LV), set at 1400 rpm. At this mixing speed, the HIPE is produced in approximately 5 minutes. In another embodiment, a HIPE is prepared using an air-powered version of the above mixer (Model 1-LAV), with air pressure set at 5-10 psi for approximately 5-10 minutes. The HIPE can be formed in a batchwise or a continuous process, such as that disclosed in U.S. Pat. No. 5,149,720 (DesMarais et al., issued Sep. 22, 1992).

Where microbeads are desired, the HIPE can be added to an aqueous solution as microdroplets, or prepared by column suspension polymerization, or via freeze-drying. The HIPE must be added to the suspension medium in an amount and at a rate suitable for forming a suspension of HIPE microdroplets. As the HIPE is added, the suspension is subjected to sufficient shear agitation to form a stable suspension. To ensure that the microbeads produced are relatively uniform in size, the mixing device used should provide a relatively uniform distribution of agitation force throughout the suspension. As shear agitation is inversely related to microdroplet size, the agitation can be increased or decreased to obtain smaller or larger HIPE microdroplets, respectively. In this manner, one can control the size of the microbead produced upon polymerization.

To produce a stable microdroplet suspension in a 22 liter spherical reactor having baffles or indents, for example, the HIPE is added to the suspension medium dropwise at a flow rate of up to about 500 ml/minute until the suspension comprises up to about 50% HIPE. Agitation can range from about 50 to about 500 rpm when a propeller- or paddle-style impeller with a diameter of approximately 1.5 to 3 inches is used. In some embodiments, the HIPE is added to the suspension medium in the 22 liter reactor at a flow rate of 20 ml/minute until the suspension comprises about 10% HIPE. Agitation of this mixture at about 250 rpm, followed by polymerization, yields microbeads with an average diameter ranging from about 100 to about 160 μm.).

Mold formation is preferred for larger size particles (greater than four, five or six mm in the smallest diameter). Once formed, the HIPE can be added to a mold form through any suitable technique, for example using a transfer apparatus such as a syringe, or by carefully pouring the emulsion into the mold cavities. The mold can have one or more predetermined shapes for forming particles of the desired shape and/or size.

Once a stable HIPE is obtained and suspended or placed into a mold, the emulsion can be polymerized by any suitable method, e.g. by heating, by photoactivation of a light-sensitive initiator, chemical free radical generation, redox initiators etc. For example, to initiate polymerization by heating, the temperature of the HIPE is increased above ambient temperature, for example by heating a mold containing the HIPE or heating the solution containing a suspension of HIPE microdroplets. Any appropriate heating method can be used, for example contacting the HIPE with a heat source, electrical heating, fuel burning, infrared light, adding the precursor material to a heated solution, etc. Polymerization conditions vary depending upon the composition of the HIPE. For example, the monomer or monomer mixture and the polymerization initiator(s) are particularly important determinants of polymerization temperature. Furthermore, the conditions must be selected such that a stable HIPE can be maintained during the time necessary for polymerization. The determination of a suitable polymerization temperature for a given HIPE is within the level of skill in the art. In general, the temperature should not be elevated above 85° C. because high temperatures can cause the emulsion to break. In one example, when AIBN is the oil-soluble initiator and potassium persulfate is the water-soluble initiator, styrene monomers are polymerized by maintaining a suspension of HIPE microdroplets at 60° C. overnight (approximately 18 hours).

The cavities in the resulting particles reflect the presence of the included aqueous discontinuous phase present during polymerization. Due to surface tension effects, the included aqueous phase droplets form a generally spherical shape, reflected in the cavities present in the resulting polymer. The diameter of an internal cavity (not adjacent to the particle surface) varies on average less than 50% in all measurable dimensions, and preferably varies less than 40%, less than 35%, less than 30%, less than 25%, less than 20%, less than 15%, or less than 10%. The diameter can be measured through scanning electron microscopy A dispersing agent may be included in suspension medium-based methods to bias the microbead shape towards a spherical shape as compared to an ellipsoidal or other nonspherical shape.

In some embodiments, the adjacent cavities are interconnected on average by a plurality of pores of smaller size than the cavities; the pores form generally circular connections between cavities, and have been observed to form one or more subpopulations of pores of generally similar sizes. In some embodiments, the cavities comprise at least six interconnecting pores on average. In some embodiments, the average interconnecting pore diameter is at least 0.5 microns. In some embodiments, the average interconnecting pore diameter is 20% or less than the average cavity diameter. In some embodiments, the ratio of average sphere or cavity size to the size of the average interconnecting pore when measured by scanning electron microscopy is of the order of 7:1.

The mechanism by which pores form in the thin-walled cavities is not fully understood. However, experimental work suggests that it is related to the quantity of porogen present and its compatibility with the cross-linked polymer and, hence, also, to the degree of cross-linking in the polymer. It is thought that prior to polymerization the high internal phase emulsion consists of three main elements: monomer and porogens in the continuous phase and water in the internal phase. The continuous phase, which consists of a homogeneous solution of porogen and the monomer and cross-linking agent and, in this situation, the porogen is compatible with the monomer mixture. It is thought that at this stage there are no interconnecting holes present in the external phase. During polymerization chain propagation takes place and as the porogen is not polymerizable and has no reactive sites in its structure, it cannot take part in polymerization. As a result, the porogen molecules separate because the porogen is no longer compatible with the growing polymeric structure and is also insoluble in the water phase. Due to the nature of a porogen, the aggregated molecules of porogen remain part of the continuous phase and probably cause the production of weak spots and subsequent pore formation in the cross-linked polymer.

Once polymerized, the porous particles are generally washed to remove any undesired remaining components after polymerization. The particles can be washed with any liquid that can solubilize such components without affecting the stability of the particle. More than one cycle of washing may be required. In one washing regimen, the particle is washed five times with water, followed by acetone extraction for roughly a day in a Soxhlet extractor. The particles can then be dried through any suitable technique; a number of methods are known in the art. In some embodiments, the particle is air-dried for two days or is dried under vacuum at 50° C. overnight.

Removal of Residual Surface Material from HIPE-Derived Particles

In some cases, some residual surface material (or skin) may remain on the surface of HIPE particles after polymerization. Shapes formed by prior art methods such as described by Barby (U.S. Pat. No. 4,522,953) yield a “skin” at the interface between the particle and the mold surface. In certain cases, materials in the suspension media used for suspension polymerization can also become incorporated into resulting polymeric microbeads. This can decrease the overall porosity of these materials, and can lead to undesired variability between batches. Therefore, it was desired to develop techniques to reduce residual surface material and improve porosity.

We have developed procedures for reducing residual surface material. In most cases, following the procedures described herein, most if not all residual surface material occurring on polymerized HIPE particles can be removed.

By “free” or “substantially free” of residual surface material or skin is meant that at least 50% of the particles when viewed by scanning electron microscopy (SEM) exhibit no observable material occluding the surface of a given porous particle structure. Preferably at least 70%, more preferably at least 80%, at least 90% or at least 95% of the particles in a population lack observable residual surface material by SEM.

The methods provided comprise treating the polymerized porous material with a surface material disrupting agent under conditions that permit the agent to disrupt the residual surface material on the polymer and increase surface porosity. The treated particles are then recovered. The methods are of particular use where agents comprising amide linkages are retained on the surfaces of polymerized particles, for example in suspension polymerization methods which use a stabilizing agent comprising amide linkages.

Any suitable surface agent disrupting material that can reduce the amount of residual surface material on a polymerized particle can be used. Of particular interest in this regard are agents that are small molecules (a molecular mass of less than 500 Daltons), or combination of small molecules, that can disrupt residual surface material on the porous polymer of interest. The surface material disrupting agent may be an oxidizing agent, for example a peroxide. Exemplary surface-material reducing agents include peroxides, anhydrides, or suitable combinations thereof. Exemplary peroxides include hydrogen peroxide and sodium peroxide. Organic peroxides such as tertiary butyl hydroperoxide, cyclohexanone peroxide, dicumyl peroxide, and the like can also be used, if desired. Hydrogen peroxide is an especially preferred oxidant and can be used in the form of an aqueous solution containing 10% to 60% hydrogen peroxide, for example 30% hydrogen peroxide. Specific surface agent disrupting materials of interest include hydrogen peroxide, succinic anhydride, and combinations thereof. Residual surface materials that can be desirably reduced by these methods include amide-containing materials, for example proteinaceous materials, including biopolymers, for example gelatin. Of interest are erodible stabilizing agents used for suspension polymerization and can become incorporated in residual surface material on a porous polymeric particle.

In some embodiments, an improved process is provided involving treatment of the porous particles for a period of time of less than 24 hours to produce particles with reduced residual surface material. In such embodiments, the temperature is raised to a temperature from about 55° C. to about 95° C., and the pH is raised to at least about 9, and may be raised up to a pH of about 12, and the particles are treated preferably for a period of time up to 24 hours. In some embodiments, however, the particle may be treated for up to about 48 hours, 72 hours, 96 hours or 120 hours. The particles can be treated for at least three, at least four, at least six, at least eight, at least ten, least 12, or at least 16 hours to reduce residual surface material. In some embodiments, the particles can be treated for up to 8, 10, 12, 15 or 18 hours to reduce residual surface material using these techniques. In some embodiments, the pH used may be about 10, about 11, or any pH from 9 to 12. The temperature used may be any temperature from about 55° C. to about 95° C., and may be at least 55° C., 60° C., 65° C., 70° C., 75° C., 80° C., 85° C., or 90° C. The temperature may be less than about 95° C., 90° C., 85° C., 80° C., 75° C., 70° C., 65° C., or 60° C.

### Methods of Use

Highly porous particles are useful for a variety of applications, notably, as an absorbent material, as solid supports in biotechnology applications, or as a carrier of active ingredients or other formulated compounds. A microbead- or other particle-based absorbent can be used, for example, to transport solvents, to absorb body fluids, and as an adhesive microcarrier. Biotechnology applications include chromatographic separations, solid phase synthesis, immobilization of antibodies or enzymes, and microbial and mammalian cell culture as well as tissue engineering. The basic microbead can be modified in a variety of ways to produce microbeads that are specialized for particular applications.

Various modifications of HIPE polymers have been described. For instance, U.S. Pat. No. 4,536,521 (Haq, issued Aug. 20, 1985) describes HIPE polymers that can be sulfonated to produce a material that exhibits a high capacity for absorption of ionic solutions. Other functionalized HIPE polymers prepared by a similar process have been described in U.S. Pats. Nos. 4,611,014 (Jomes et al., issued Sep. 9, 1986) and 4,612,334 (Jones et al., Sep. 16, 1986), both incorporated herein by reference.

Functionalized particles including microbeads can be produced by known methods and are disclosed, for example, in U.S. Pat. No. 4,611,014 (Jomes et al., issued Sep. 9, 1986), incorporated by reference in its entirety. Briefly, the functionalized particle can be prepared indirectly by chemical modification of a preformed microbead bearing a reactive group such as bromo or chloromethyl. Particles suitable for subsequent chemical modification can be prepared by polymerization of monomers such as chloromethylstyrene or 4-t-BOC-hydroxystyrene. Other suitable monomers include styrene, α-methylstyrene, or other substituted styrene or vinyl aromatic monomers that, after polymerization, can be chloromethylated to produce a reactive intermediate that can be subsequently converted to a functional group of interest. The concentration of the reactive monomer should generally be sufficiently high to ensure that the functionalized particle generated after chemical modification bears the desired functional groups e.g. ionic or polar) on a minimum of about 30% of the monomer residues.

Chemical modification of the reactive particle intermediate is carried out by any suitable technique. Exemplary methods for producing amine-, amine salt-, and cationic quaternary ammonium-functionalized microbeads are described in detail in the Examples.

In other embodiments, microbeads bearing ionic or polar groups can be prepared directly by emulsification and polymerization of an appropriate substantially oil-soluble monomer.

### Production of a Carbonized HIPE-Derived Particle

A particle treated to remove residual surface material as described herein, can be further converted to a porous carbonized material that retains the original internal structure of cavities and interconnecting pores. Carbonized particles are useful for a wide variety of applications, for example, as a sorption or filtration medium and as a solid support in a variety of biotechnology applications, as described further herein. In addition, the carboniferous particles can be used as an electrode material in batteries, super-capacitors and other devices utilizing electrochemical conversion processes; the large lattice spacing in the HIPE-derived particle is particularly in this regard. A large lattice spacing reduces or eliminates lattice expansion and contraction during battery operation, extending battery cycle lifetimes. HIPE-derived carbonized particles are ideally suited for super-capacitors, which require highly conductive electrodes, because carbon is an excellent conductor and the interconnectedness of the particle maintains continuity of electrical connections.

The carbonized particles thus produced can be used in any application requiring electrochemical conversion, including in fuel cell and related applications requiring catalysis, as they are highly effective conductors of electricity. Thus, carbonized microbeads can be used to support platinum, a platinum alloy or another appropriate catalyst to transfer electrons resulting from oxidation of hydrogen gas, methanol or other reactive material brought to the surface of the carbon-catalyst structure. Current can then travel through a circuit and provide electrical power.

Catalysts can be deposited on the carbonized particles by appropriate means to form a catalyst-carbon surface useful in a variety of catalytic reactions. The porous nature of the carbonized structure provides pathways for the catalytic materials to be deposited throughout the carbonized particle. Any method useful for depositing a catalyst or conductive metal to the surface of the particle which does not preclude the intended use of the resulting particle can be used. For example, colloidal suspensions of platinum can be used to deposit platinum on the carbonized particles by means of an appropriate carrier solution. Catalysts may be sputtered on the particles by means known in the art. Exemplary catalysts include platinum, palladium, and alloys of either thereof. In some embodiments, platinum, platinum alloys or other appropriate catalysts can be added to the surface of the polymer microbead prior to treatment of the polymer microbead in the furnace used to produce the carbonized structure. Following treatment in the furnace, the platinum group metal, alloy or other appropriate catalyst remains on the surface or becomes embedded in the resulting carbonaceous structure. This resulting carbon-catalyst structure is useful in carrying out electrochemical conversions such as in fuel cells or other catalyzed reactions.

To produce a carbonized particle, the particle is heated in an inert atmosphere as disclosed for HIPE polymers in U.S. Pat. No. 4,775,655 (Edwards et al., issued Oct. 4, 1988), which is incorporated herein by reference in its entirety.

The ability of the particle to withstand this heat treatment varies depending on the monomer or monomers used. Some monomers, such as styrene-based monomers, yield microbeads that must be stabilized against depolymerization during heating. The modification can take many forms. Polymer components and process conditions can be selected to achieve a high level of cross-linking or to include chemical entities that reduce or prevent depolymerization under the heating conditions employed. Also, suitable stabilizing chemical entities can be incorporated into or added to the polymer, including halogens, sulfonates, chloromethyl, methoxy, nitro, and cyano groups. For maximum thermal stability, the level of cross-linking is preferably greater than about 20% and the degree of any other chemical modification is at least about 50%. Stabilizing entities can be introduced into the microbead after its formation or by selection of appropriately modified monomers. Once stabilized, the microbead is heated in an inert atmosphere to a temperature above 500° C. To reduce the stabilizer content of the final carbonized structure, the temperature should generally be raised further, for example to about 1200° C.

### Loading Substances Into the Particles

The utility of the particle can be increased by loading a gel or other formulated material to the particle interior according to the methods described in U.S. Pat. No. 4,965,289 (Sherrington, issued Oct. 23, 1990). The gel can be formed in or added to the particle cavities and may be linked to the particle surface. In some embodiments, the gel may bear either acidic or basic groups, depending on whether the particle substrate is to serve as an anion-exchange resin or a cation-exchange resin, respectively.

Other substances can be loaded into the particle according to intended applications. For example, various cosmetics or skin care formulations can be added to the particles. Particles prepared according to this invention are amenable to incorporation of gels or other substances due to the improved surface porosity. Exemplary cosmetics suitable for loading into the particles include those sold by Johnson and Johnson, Pierre Fabre, Chanel, Este Lauder, and others.

### Use of the Particles in Cell Culture and Tissue Engineering

In addition to the above applications, the particle is also useful in cell culture. High density cell culture generally requires that cells be fed by continuous perfusion with growth medium. Suspension cultures satisfy this requirement; however, shear effects limit aeration at high cell concentrations. The particle protects cells from these shear effects and can be used in conventional stirred or airlift bioreactors.

To prepare a particle for use in culturing eukaryotic or prokaryotic cells, the particle is typically sterilized by any available sterilization methods. Suitable methods include irradiation, ethylene oxide treatment, and, preferably, autoclaving. Sterile polymeric structures are then placed in a culture vessel with growth medium suitable for the cells to be cultured. Suitable growth media are known. An inoculum of cells is added and the culture is maintained under conditions suitable for cell attachment to the particles. The culture volume is then generally increased, and the culture is maintained in the same manner as prior art suspension cultures.

Polymers thus produced can be used in cell culture or tissue engineering without modification; however, the particles can also be modified to improve cell attachment, growth, and the production of specific proteins. For instance, a variety of bridging molecules can be used to attach cells to the microbeads. Exemplary bridging molecules include antibodies, lectins, glutaraldehyde, polycationic species (e.g., poly-L-lysine), and/or matrix or basement membrane molecules (fibronectin, vitronectin, thrombospondin, collagen, etc.). In addition, sulfonation of particles can increase cell attachment rates in some instances. Inoculating particles with cells for cell culture or tissue engineering is greatly enhanced using particles prepared according to this invention since substantially all the surface is porous and available for loading or inoculation.

### Use in Drug Delivery Applications

Particles prepared by the described techniques can be used to deliver drugs such that near zero-order kinetics are realized. In one example, Naprosyn is dissolved in melted polyethylene glycol (PEG) and the microbeads are allowed to absorb this liquid. Excess coating of the PEG-Naprosyn mixture is washed from the beads. A drug release profile is obtained by placing the beads containing the PEG-Naprosyn mixture into a beaker containing water or PBS and stirred with a magnetic stirrer. An aliquot of solution is taken periodically over 24 hours.

### EXAMPLES

The invention is further illustrated by the following specific but non-limiting examples.

### Example 1

### Removal of Residual Surface Material Using Succinic Anhydride

Poly(styrene-divinylbenzene) HIPE microbeads are prepared via suspension polymerization using gelatin as a stabilizing agent (Li et al., U.S. Pat. Nos. 5,583,162; 5,653,922; 5,760,097; 5,863,957; 6,100,306). Ten grams of these microbeads (wet) are placed into a 2 liter glass reactor and 1.0 liter of distilled water in which 30 grams of succinic anhydride (97% from Aldrich) is dissolved is added under stirring. Approximately 20 grams of sodium hydroxide is then added into the mixture and the pH of the system is kept at 10-11. The temperature of the reaction is kept at 55-60° C. for at least 4 hours. The microbeads, now substantially free of residual surface material, is then washed and dried according to Li et al.

### Example 2

### Removal of Residual Surface Material Using Hydrogen Peroxide

The microbeads are prepared as described in Example 1. Ten grams of the microbeads are placed into a two liter reactor and 1.0 liters of 3% aqueous hydrogen peroxide is added. The pH of the mixture is kept at 8-9, adjusted using 2% aqueous sodium hydroxide. This system is kept at 55-60° C. for 24 hours under mechanical stirring. The treated microbeads are then washed and dried.

### Example 3

### Preparation of Polyvinylalcohol Microbeads

### Example 3A

### Preparation of PVA Using Vinyl Pivalate

Polyvinylalcohol (PVA) microbeads were prepared according to the following protocol. The final concentration of each component of the HIPE and the aqueous suspension medium are shown in Tables 1 and 2.

1. Prepare a continuous phase by combining vinyl pivalate monomer, divinylbenzene (DVB), Span 80, AIBN, toluene, calcium chloride, and 2480 mL water with stirring at room temperature.

2. Prepare an aqueous discontinuous phase by adding 5 grams potassium persulfate to 2480 mL of deionized water.

3. Stir the continuous phase at approximately 3500 rpm, and then add the aqueous discontinuous phase to the continuous phase at a flow rate of 20 ml/minute. Stir the combined phases at 3500 rpm for approximately 5-10 minutes to form a stable HIPE.

4. Prepare an aqueous suspension medium by combining potassium persulfate and gelatin with the deionized water. Stir the mixture at 300 rpm for about 15 minutes.

5. Add the HIPE to the aqueous suspension medium dropwise at a flow rate of 15 ml/minute in a 22 liter Lurex reactor until the suspension reaches about 20% HIPE.

6. To form microbeads, polymerize the suspension by raising the temperature to 67±2° C. for twenty-four hours while stirring at 300 rpm.

7. Wash the resultant microbeads five times with water and then perform acetone extraction in a Soxhlet extractor for about a day. Allow the microbeads to air-dry overnight. The density of the resultant material is 0.07 gm/ml of dried microbeads.

8. Post-treatment of polymer beads:
a) The beads are passed through standard sieves to obtain beads having the desired size distribution. Exemplary size ranges include: 38 to 106 micrometers, 106 to 250 micrometers, 250-425 micrometers and above 425 micrometers (by using U.S.A. Standard test sieve; sieve sizes are 38 μm, 106 μm, 250 μm and 425 μm).
b) The sieved beads are washed five times with hot water (60° C.), followed by five acetone washes.
c) The washed beads are contacted with 300 g succinic anhydride (Aldrich, 134414) and 300 g of NaOH (Sigma, 221465) dissolved in 10 liters of distilled water. Enough NaOH is added to the mixture sufficient compatible base to achieve a pH of 12. The mixture is then stirred at 300 RPM overnight at 65° C.
d) The beads are then filtered and washed with water until a pH of about 6 to about 7 is achieved. The beads can then be suspended in water for verification of removal of residual surface material as well as to determine particle size by SEM testing.
e) Hydrolyze the beads using 4N NaOH (adjust pH>9) at 70° C. for 40 hrs.
f) The polymer beads can be filtered and purified, for example, using a Soxhlet Extractor to extract soluble residues, for example using acetone as the extraction solvent. Extraction is continued for approximately 48 hours or until no further extractable soluble residues are detected. The products are then air dried at room temperature first, then oven dried under vacuum overnight.

9. FINAL WASH AND DRY by using water, methanol and acetone; then dry at 60° C. RESULT: After hydrolysis, the beads have a slightly yellow color.

TABLE 1
Preparation of HIPE (80%)

| Component | Amount | |
|---|---|---|
| Vinyl pivalate | 277 | g (320 mL) |
| Divinylbenzene (55%) | 90 | g (100 mL) |
| Toluene | 173 | g (200 mL) |
| AIBN | 4.8 | g |
| Span 80 | 80 | g |
| Water (deionized) | 2480 | mL |
| CaCl₂ 6H₂O | 74.7 | g |
| K₂S₂O₈ | 5 | g |

TABLE 2
Suspension Medium

| Component | Amount |
|---|---|
| Water (deionized) | 10 L |
| Gelatin | 330 g |
| K₂S₂O₈ | 15 g |
| CaCl₂ 6H₂O | 500 g |

### Example 3B

### Preparation of PVA Using Vinyl Propionate

All amounts and conditions the same as in Tables 1 and 2; however vinyl propionate is substituted for vinyl pivalate.

### Example 4

### Production of Polymethyl Methacrylate (PMMA) Microbeads

1. Prepare a continuous phase by combining 300 g of methyl methacrylate monomer, 120 g of divinylbenzene (DVB), 84 g of Span 80, 5.25 g of AIBN, 172 g of toluene and 81 g of calcium chloride with stirring at room temperature.

2. Prepare an aqueous discontinuous phase by adding 5 grams potassium persulfate to 3690 mL of deionized water.

3. Stir the continuous phase at approximately 3500 rpm, and then add the aqueous discontinuous phase to the continuous phase at a flow rate of 20 ml/minute. Stir the combined phases at 3500 rpm for approximately 15 minutes to form a stable HIPE.

4. Prepare an aqueous suspension medium by combining potassium persulfate and gelatin with the deionized water. Stir the mixture at 700 rpm for about 15 minutes, and then adjust the stirring speed to 295-300 rpm.

5. Add the HIPE to the aqueous suspension medium dropwise at a flow rate of 15 ml/minute in a 22 liter Lurex reactor.

6. To form microbeads, polymerize the suspension by raising the temperature to 70±1° C. for twenty-four hours while stirring at 300 rpm.

7. Wash the resultant microbeads five times with water and then perform acetone extraction in a Soxhlet extractor for about a day. Allow the microbeads to air-dry overnight. The density of the resultant material is 0.10-0.12 gm/ml of dried microbeads.

8. Post-treatment of polymer beads:
a) Pass the beads through standard sieves to obtain beads having the desired size distribution. For example, in the following ranges: 38 to 106 micrometer, 106 to 250 micrometer, 250 to 425 micrometer and above 425 micrometer (by using U.S.A. Standard test sieve; sieve sizes are 38 μm, 106 μm, 250 μm and 425 μm).
b) Wash the obtained beads, five times, with hot water (60 C), followed by an acetone wash, five times.
c) To remove residual surface material from each set of prepared beads, mix 300 g succinic anhydride (Aldrich, 134414) with 300 g of NaOH (Sigma, 221465) (the NaOH will be dissolved into water in situ) and beads, in 10 liters of distilled water. Add enough NaOH to the mixture to adjust the pH to 9-12. Stir the mixture at 300 RPM overnight, at 65 C.
d) Then filter the beads and wash with water to get pH of 6-7. Polymer beads are then suspended in water to review treatment results as well as particle size by using SEM testing.
e) The classified polymer beads are filtered and finally are purified by using a Soxhlet Extractor to extract any soluble residue using acetone as the extraction solvent. This solvent extraction is continued for about 2 days and/or until no residual chemicals are detected in the extract. The products are then dried first at room temperature, then in a vacuum oven overnight.

9. FINAL WASH AND DRY by using water, methanol and acetone; then dry at 60° C.

### Example 5

### Functionalization of Particles for Acid Absorption Using Beads Modified with Amine Groups

Diethylamine-functionalized particles are produced from chloromethyl styrene particles prepared as described in Li et al., however, before functionalization, the skin is removed by appropriate means as described in this specification. The particles are air-dried overnight and Soxhlet extracted for 15 hours with 200 ml hexane to remove residual unpolymerized components. 5 gm of particles are then refluxed with 150 ml aqueous diethylamine for 20 hours.

### Example 6

### Functionalization of Particles for Acid Absorption Using Amine Salts

To produce a dihexylammonium salt, dihexylamine-functionalized particles are prepared as described above in Example 7 for diethylamine-functionalized particles. 1 gm dihexylamine-functionalized particles are then added to 100 ml methanolic HCl and stirred for 30 minutes. The counterion of the resultant salt is chloride. The dihexylammonium chloride-functionalized particles are collected by filtration, washed with 3 times with 50 ml methanol, and air-dried overnight.

### Example 7

### Functionalization of Particles for Absorption of Acids Using Quaternary Ammonium Groups

To produce a dimethyldecylammonium salt, chloromethylstyrene particles are prepared according to Li et al., and residual surface material is removed according to Example 1. The particles are air-dried overnight and Soxhlet extracted with hexane to remove residual unpolymerized components. 1 gm particles are then filled under vacuum with a 10-fold molar excess of ethanolic amine and refluxed for 7 hours. The counterion of the resultant salt is chloride. The dimethyldecylammonium chloride-functionalized particles are collected by filtration, washed twice with 50 ml ethanol and twice with 50 ml methanol, and then air-dried overnight.

### Example 8

### Functionalization of Particles for Absorption of Aqueous Solutions Using Amine Salts

To produce a dimethylammonium salt, diethylamine-functionalized particles are prepared as described in Example 6. The particles are air-dried overnight and Soxhlet extracted with hexane to remove residual unpolymerized components. 1 gm particles are then added to 100 ml methanolic HCl and stirred for 30 minutes. The counterion of the resultant salt is chloride.

### Example 9

### Functionalization of Particles for Aqueous Absorption Using Quaternary Ammonium Groups

To produce a dimethyldecylammonium salt, chloromethylstyrene particles are prepared as described in Example 1. The particles are air-dried overnight and Soxhlet extracted with hexane to remove residual unpolymerized components. 1 gm particles are then treated with 100 ml aqueous amine for 30 minutes.

### Example 10

### Functionalization of Particles for Absorption of Aqueous Solutions Using Alkoxylate Groups

Ethoxylated particles are prepared from chloromethylstyrene particles prepared as described in Example 7. The particles are air-dried overnight and Soxhlet extracted with hexane to remove residual unpolymerized components. 1 gm particles are then treated with 100 ml of an anionic form of a polyethylene glycol (PEG) containing 8-9 ethylene glycol monomers in excess PEG as solvent. The reactants are heated at 95° C. for 2 hours.

### Example 11

### Functionalization of Particles for Absorption of Aqueous Solutions Using Sulfonate Groups

Sulfonate-functionalized particles are produced from styrene particles prepared as described in Example 1. The particles are dried under vacuum at 50° C. for two days. 10 gm of particles were then added to a 500 ml flask containing a mixture of 200 ml of chloroform and 50 ml of chlorosulfonic acid. The flask is shaken at room temperature for two days. The sulfonate-functionalized particles are collected by filtration and washed sequentially with 250 ml each of chloroform, methylene chloride, acetone, and methanol. The particles are soaked in 300 ml 10% aqueous sodium hydroxide overnight and then washed with water until the eluate reaches neutral pH.

### Example 12

### Production of Gel-Filled Particles for Use as a Substrate for Protein Synthesis

Particles with a void volume of 90%, a density of 0.047 gm/cm, an average cavity diameter in the range of 1-50 μm, and which are 10% cross-linked are prepared as described in Example 1. The gel employed is poly(N-(2-(4-acetoxyphenyl)ethyl)-acrylamide). To produce a solution of gel precursors, 2.5 gm of monomer, 0.075 gm of the crosslinking agent ethylene bis(acrylamide), and 0.1 gm of the initiator AIBN is added to 10 ml of the swelling agent dichloroethane. The gel precursor solution is then deoxygenated by purging with nitrogen.

0.7 gm of particles is added to the gel precursor solution and polymerization is initiated by heating the mixture at 60° C. while rotating the sample on a rotary evaporator modified for reflux. The dichloroethane swells the particles, allowing the gel precursors to penetrate the particle and form a polyamide that becomes interpenetrated with the polymer chains of the particle. After 1 hour, the gel-filled particles (hereinafter “composite”) are washed with 50 ml dimethyl formamide (DMF) and 50 ml diethyl ether and then vacuum dried.

To produce chemical groups within the composite, 0.25 gm of the composite is treated with 50 ml of a 5% solution of hydrazine hydrate in DMF for 5 minutes. This treatment provides free phenolic functionalities within the gel matrix that act as chemical groups for synthesis.

### Example 13

### Use of Particles in High Density Cell Culture

To produce particles suitable for mammalian cell culture, sulfonated particles are prepared as described in Example 12 and are then wetted in a 70% ethanol solution and autoclaved at 121° C. for 15 minutes. The particles are then washed twice with sterile phosphate-buffered saline and once with complete growth medium. 500 mg of the sterile particles are placed in a 500 ml roller bottle that has been siliconized to prevent attachment of the cells to the bottle.

An inoculum of 5×10⁷ baby hamster kidney cells in 50 ml of growth medium (containing 10% fetal calf serum) is added to the roller bottle. The inoculum is incubated with the particles for 8 hours at 37° C. with periodic agitation to allow cell attachment to the particles. The culture volume is then increased to 100 ml, and the roller bottle is gassed with an air-CO₂ (95:5) mixture and placed in a roller apparatus. Growth medium is replaced whenever the glucose concentration drops below 1 gm/liter.

### Example 14

### Production of Stable Carbon Structure from Sulfonated Particles

To produce stable carbonaceous structures, sulfonated particles are first prepared according to Example 12 such that the level of cross-linking is between 20% and 40% and the void volume is 85%. The dried, sulfonated particles are then placed in an electrically heated tube furnace and the temperature is increased to 600° C. in an oxygen-free nitrogen atmosphere. The rate of heating is generally maintained below 5° C. per minute and in the range of 180° C. to 380° C., the rate of heating does not exceed 2° C. per minute. After the heating process, the particles are cooled to ambient temperature in an inert atmosphere to prevent oxidation by air.

1-34. (canceled)

35. A method of removing residual surface material from a porous polymeric particle comprising cavities linked by interconnecting pores,
comprising contacting the porous polymeric particle with a surface material disrupting agent at a pH to about 9 to about 12 and a temperature of from about 55-95° C. for a period of from about 4 to about 24 hours, to remove residual surface material from the surface of the particle under conditions that permit the agent to disrupt the surface material; and
recovering the polymerized porous particle having improved surface porosity.

36. The process of claim 35, wherein the surface material comprises gelatin.

37. The process of claim 35, wherein the surface material disrupting agent comprises a peroxide, an anhydride, or a combination thereof.

38. The process of claim 35, wherein the surface material disrupting agent is selected from hydrogen peroxide and succinic anhydride.

39. The process of claim 35, wherein the polymeric particle is prepared by suspension polymerization.

40. The process of claim 39, wherein the suspension polymerization is performed using an erodible stabilizing agent in the suspension medium.

41. The process of claim 35, wherein the polymeric particle is a microbead.

42. The process of claim 35, wherein the polymeric particle is prepared by polymerization of a high internal phase emulsion (HIPE).

43. The process of claim 35, wherein at least 70%, at least 80%, at least 90%, or at least 95% of the treated particles are free of residual surface material as determined by scanning electron microscopy.

44. The process of claim 35, wherein the polymerized porous particle is prepared using an optionally derivatized vinyl monomer selected from vinyl, vinyl chloride, styrene, acrylic acid, an acrylic acid ester, vinyl alcohol, and a vinyl alcohol ester.

45. The process of claim 43, wherein the optionally derivatized vinyl monomer is selected from styrene, methyl methacrylate, vinyl pivalate, and vinyl propionate.

46. The process of claim 35, wherein the polymerized porous material is prepared using one or more optionally derivatized crosslinking agents selected from the group consisting of a divinyl compound, a trivinyl compound, a diacrylic compound, a triacrylic compound, triallyl isocyanurate, and a combination thereof.

47. The process of claim 45, wherein the optionally derivatized crosslinking agent is divinylbenzene.

48. A porous crosslinked polymeric particle produced by the process of claim 35.

49. The particle of claim 48, wherein the particle has a void volume of at least 75%, at least 80%, at least 85%, at least 90%, at least 95%, or at least 97%.

50. The particle of claim 48, wherein the particle has a measured density of less than about 0.20 gm/cm³ or less than about 0.10 gm/cm³.

51. The particle of claim 48, wherein at least 50%, at least 60%, at least 70%, at least 80%, at least 85%, at least 90%, or at least 95% of the cavities at the interior of the particle communicate with the surface of the particle.

52. The particle of claim 48, wherein the cavity size is in the range of about 1 to about 50 microns in diameter, wherein the cavities comprise on average a plurality of pores in walls separating adjacent cavities.

53. The particle of claim 48, wherein the average interconnecting pore diameter is 20% or less of the average cavity diameter.

54. The particle of claim 48, wherein the particle is modified so that:
the particle is functionalized;
the particle is carbonized;
the particle has a metal and/or catalyst deposited throughout the particle;
the particle has a gel or pre-gel deposited within the particle cavities; and/or
the particle has a chemical, pharmaceutical, cosmetic, formulation or combination thereof deposited within the particle cavities.
